(19) United States
(12) Patent Application Publication
(10) Pub. No.: US 2002/0002319 A1
(43) Pub. Date: Jan. 3, 2002

(54) PROCESS FOR ISOMERISING C5-C8 PARAFFIN CUTS RICH IN PARAFFINS CONTAINING MORE THAN SEVEN CARBON ATOMS

(76) Inventors: ERIC BENAZZI, CHATOU (FR); HERVE CAUFFRIEZ, BOUGIVAL (FR); OLIVIER CLAUSE, CHATOU (FR); JEAN-FRANCOIS JOLY, LYON (FR); CHRISTINE TRAVERS, RUEIL MALMAISON (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/199,350

(22) Filed: Nov. 25, 1998

(30) Foreign Application Priority Data
Nov. 25, 1997 (FR) 97/14.892

(51) Int. Cl. C07C 5/13

(52) U.S. Cl. 585/748; 585/747; 585/750; 585/751; 585/734

(57) ABSTRACT

A process for isomerising a feed comprising normal paraffins containing 5 to 8 carbon atoms per molecule as a major portion in the presence of hydrogen is characterized in that the sum of the amounts of normal paraffins containing 7 and 8 carbon atoms per molecule contained in the feed is in the range 2% to 90% by weight with respect to the feed, and in that said feed is treated in at least one reaction zone containing at least one catalyst in a fixed bed, said catalyst comprising a support, at least one halogen and at least one group VIII metal, the reaction being carried out at a temperature in the range 30° C. to 150° C.

The present invention relates to a process for isomerisation, in the presence of hydrogen (sometimes also known as a hydroisomerisation process) of a feed comprising normal paraffins (also termed n-paraffins or normal paraffins) containing 5 to 8 carbon atoms per molecule as a major portion.

The removal of lead alkyls from automobile gasoline for environmental protection reasons has prompted the development of processes for producing branched chain paraffins which have a better octane number than linear compounds, in particular a process for isomerising normal paraffins to branched paraffins. The importance of this process to the petroleum industry id currently increasing.

Isomerising n-butane (normal butane) leads to the production of isobutane which can be used in different applications. Examples of such applications are: processes for alkylating light olefins to produce paraffinic cuts containing 5 to 12 carbon atoms per molecule. Alkylation is carried out using at least one isoparaffin, and it can produce cuts with high octane numbers. After dehydrogenation, isobutane can also be used to etherify methanol or ethanol. The ethers obtained—methyl tertio-butyl ether (MTBE) or ethyl tertio-butyl ether (ETBE) have high octane numbers and can be directly incorporated into the gasoline.

The process for isomerising paraffins containing 5 and 6 carbon atoms per molecule can also lead to the production of high octane number gasoline bases which can be directly incorporated into the gasoline fractions. The latter process has been the subject of numerous studies, and three different catalyst types have traditionally been used to carry out the isomerisation reaction:

Friedel-Crafts type catalysts, such as catalysts containing aluminium chloride, which are used at low temperatures (about 20° C. to 130° C.);

catalysts based on metals from group VIII of the periodic table (“Handbook of Chemistry and Physics”, 45^{th} Edition, 1964-1965) deposited on alumina and generally containing a halogen, which are used at medium temperatures (about 110° C. to 160° C.). U.S. patents U.S. Pat. No. 2,906,798, U.S. Pat. No. 2,993,398, U.S. Pat. No. 3,791,960, U.S. Pat. No. 4,113,789, U.S. Pat. No. 4,149,993, U.S. Pat. No. 4,804,803, for example, describe such catalysts;

zeolitic catalysts comprising a group VIII metal deposited on zeolite, used at high temperatures (250° C. to 350° C.); such catalysts lead to the production of a mixture of hydrocarbons with an octane number which is improved but is not as good as that obtained by processes using the catalysts cited above. However, they have the advantage of being easier to use and more resistant to poisons. The low acidity does not enable them to be used to isomerise n-butane. U.S. Pat. No. 4,727,217 describes such catalysts.

Current processes for isomerising paraffins containing 5 and 6 carbon atoms using chlorinated alumina type catalysts and including platinum are high activity catalysts. Such processes are used once through or with partial recycling after fractionating the unconverted normal paraffins, or with complete recycling after passage through molecular sieve systems in the liquid phase. Such processes lead to the production of a base for fuels containing no aromatic compounds and with a research octane number (RON) generally in the range 82 to 88, the normal paraffin isomerisation process including or not including recycling.

Numerous patents have concerned monometallic platinum based catalysts deposited on a halogenated alumina, and their use in normal paraffin isomerisation processes. U.S. Pat. No. 3,963,643 describes treatment with a Friedel-Crafts type compound followed by treatment with a chlorinated compound containing at least two chlorine atoms, such treatment more particularly being applicable to straight chain hydrocarbons containing 4 to 6 carbon atoms. U.S. Pat. No. 5,166,121 describes a catalyst comprising gamma alumina in the form of beads and comprising 0.1% to 3.5% of halogen on the support. The amount of halogen (preferably chlorine) deposited on the support is extremely small.

As is currently publicly known, a major drawback of such processes is that they feeds containing more than about 2% by weight of normal paraffins containing at least 7 carbon atoms per molecule cannot be treated properly. Operating conditions which are known to encourage isomerisation of cuts comprising paraffins containing 5 and 6 carbon atoms per molecule leads to degrees of cracking for paraffins containing at least 7 carbon atoms per molecule and which are too large (of the order of 20% to 80% for C7 paraffins).

An examination of the prior art shows that catalysts for isomerising normal paraffins containing 7 carbon atoms have been studied. In such processes, the reaction temperature is generally over 200° C. and normally over 300° C. and the ratio of the number of moles of hydrogen over the number of moles of hydrocarbons is more than 1. Such operating conditions do not encourage the production of highly branched paraffins. Thermodynamic equilibrium data indicate that the amount of paraffin branching reduces as the temperature increases.

French patent FR-A-2 735 993 describes a catalyst and its use in processes for isomerising normal paraffins containing 4 to 6 carbon atoms. The catalyst contains at least one halogen, preferably chlorine, at least one group VIII metal and a formed support comprising gamma alumina and/or eta alumina, the catalyst being characterized in that the smallest average dimension of said support is about 0.8 to 2 mm, preferably about 1 to 1.8 mm, and in that its chlorine content is about 4.5% to 15% by weight, preferably about 5% to 12% by weight. The catalyst is prepared by halogenating a catalyst containing at least one group VIII metal on an alumina support. Once the metal has been deposited, the support can be activated in air and/or in nitrogen.

A halogenated catalyst can also be prepared from a support which is formed and steam treated. Such a catalyst forms the subject matter of a patent application by the Applicant, filed on the same day as the present application, which describes a catalyst containing at least one halogen, at least one group VIII metal and a formed support comprising gamma alumina and/or eta alumina, treated in a stream of gas containing steam.

The present invention provides a process for isomerising a feed comprising normal paraffins containing 5 to 8 carbon atoms per molecule as a major portion in the presence of hydrogen, characterized in that the sum of the amounts of normal paraffins containing 7 and 8 carbon atoms per molecule contained in the feed is in the range 2% to 90% by weight with respect to the feed, and in that said feed is treated in at least one reaction zone containing at least one catalyst in a fixed bed, said catalyst comprising a support, at least one halogen and at least one group VIII metal, the reaction being carried out at a temperature in the range 30° C. to 150° C.

The present invention also provides a process for increasing the octane number of a petroleum cut comprising normal paraffins containing 5 to 8 carbon atoms per molecule. In particular, the present invention overcomes the above disadvantages. The process of the invention can convert feeds in which the sum of normal paraffins containing 7 and 8 carbon atoms per molecule contained in said feed is in the range 2% to 90% by weight, preferably in the range 5% to 90% by weight, more preferably in the range 20% to 90% by weight, and very preferably in the range 40% to 90% by weight. The process of the present invention can produce a yield of branched paraffins containing at least 5 carbon atoms per molecule of over 85% by weight from a feed to be treated comprising normal paraffins containing 5 to 8 carbon atoms per molecule,.

The process of the present invention uses at least one reaction zone which comprises at least one reactor preferably containing at least one solid acid catalyst in a fixed bed, the reaction temperature being in the range 30° C. to 150° C., preferably in the range 70° C. to 130° C., more preferably in the range 70° C. to 95° C. The catalyst used comprises a support, preferably an alumina based support, containing at least one halogen, the halogen content being in the range 0.1% to 15% by weight, and at least one group VIII metal. In a preferred embodiment of the invention, a chlorinated alumina based catalyst is used.

The catalyst used in the process of the invention contains at least one group VIII metal on a support, preferably an alumina based support, and on the support at least one halogen is deposited, preferably selected from the group formed by fluorine, chlorine, bromine and iodine; more preferably the halogen is chlorine. The halogen content is in the range 0.1% to 15% by weight, preferably in the range 4% to 12% by weight. The catalyst support preferably essentially comprises alumina. The alumina which is preferably used in the process of the invention can be gamma alumina and/or, possibly, eta alumina (i.e., constituted either by gamma alumina, or eta alumina, or a mixture of these two aluminas). When gamma alumina is added to eta alumina, the alumina of the support comprises between 50% and 100% by weight, preferably between 80% and 100% by weight, of eta alumina, more preferably 80% to 95% by weight of eta alumina, the complement being gamma alumina.

The smallest average dimension of the catalyst support is about 0.8 to 2 mm, preferably about 1 to 1.8 mm. The support is preferably essentially formed from beads with an average diameter of about 0.8 to 2 mm, preferably about 1 to 1.8 mm, or the support is essentially formed from extrudates with a smallest dimension of about 0.8 to 2 mm, preferably about 1 to 1.8 mm, i.e., the extrudates are formed using any extrusion technique known to the skilled person, such as a die with a diameter of about 0.8 to 2 mm, preferably about 1 to 1.8 mm.

The gamma alumina possibly present in the catalyst support has a specific surface area of about 150 to 300 m²/g and preferably about 180 to 250 m²/g, and a total pore volume of about 0.4 to 0.8 cm³/g, preferably about 0.45 to 0.7 cm³/g.

The eta alumina which is optionally present in the catalyst support has a specific surface area of about 400 to 600 m²/g, preferably about 420 to 550 m²/g, and a total pore volume of about 0.3 to 0.5 cm³/g, preferably about 0.35 to 0.45 cm³/g.

The group VIII metal is selected from the group formed by iron, cobalt, nickel, ruthenium, rhodium, palladium, osmium, iridium and platinum, preferably selected from the group formed by platinum, palladium and nickel. In the preferred case where said metal is platinum or palladium, the content is about 0.05% to 2% by weight, preferably about 0.1% to 1.5% by weight. In the preferred case where said metal is nickel, the content is about 0.1% to 10% by weight, preferably about 0.2% to 6% by weight.

The catalyst is generally prepared by forming the support. The formed support can optionally be steam treated at high temperature before or after depositing at least one group VIII metal. Halogenation, preferably chlorination, is then carried out. It is also possible, and preferred, to carry out an activation step in hydrogen before said halogenation step. Each step of the process for preparing the support of the invention will be explained below.

When two types of alumina (gamma and eta) are present in the catalyst support, these two types of aluminas are preferably mixed and formed together using any technique which is known to the skilled person, for example by extrusion through a die, pelletization or bowl granulation. However, it is also possible to form the two types of alumina separately then to proceed to mixing the two types of formed alumina. In all cases, the smallest dimension of the geometric shape described by the support after forming is about 0.8 to 2 mm, preferably about 1 to 1.8 mm, in order to produce a sufficient halogen content for a reduced halogenation period during the support halogenation step.

The support preferably undergoes high temperature treatment using steam. The hydrothermal treatment is generally carried out for 0.5 to 6 hours, for example, at a temperature of about 200° C. to 700° C. in a stream of gas, for example air and/or nitrogen. The gas must contain water, for example in an amount of about 0.2% to 100% by volume and preferably about 0.3% to 20% by volume. Activation of the alumina by the steam can produce much more acidic catalysts which are thus more active for isomerisation.

At least one hydrogenating metal from group VIII selected from the group formed by iron, cobalt, nickel, ruthenium, rhodium, palladium, osmium, iridium and platinum, preferably selected from the group formed by platinum, palladium, and nickel, is then deposited on the support using any technique which is known to the skilled person, for example by anion exchange using hexachloroplatinic acid when using platinum or in the form of the chloride when using palladium. The hydrothermal treatment can also be carried out after depositing the metal on the support.

The support comprising the deposited metal can thus optionally undergo a treatment in hydrogen to produce an active metallic phase. The procedure of this treatment under hydrogen comprises, for example, a slow rise in temperature in a stream of hydrogen up to the maximum reduction temperature which is about 300° C. to 700° C., preferably in the range 340° C. to 680° C., followed by holding that temperature generally for 1 to 6 hours, preferably for 1.5 to 4.5 hours.

The halogenation step can be carried out using any technique known to the skilled person. The halogen, preferably chlorine, is preferably deposited from any carbon-containing compound also containing halogen atoms and known to allow halogenation, preferably under conditions which the skilled person would judge to be suitable having regard to treatment of effluents, the halogenation duration or the cost. For this reason, the hydrogen chloride is rarely if ever used. Alumina is halogenated, preferably chlorinated, directly in an isomerisation unit before injecting the feed to be treated, or offsite: in a separate unit provided for halogenation. Halogenation can be carried out using any halogenating carbon-containing agent, preferably a chlorinating agent, which is known to the skilled person. In the preferred case when the halogen is chlorine, carbon tetrachloride or chloroform are normally used.

In the catalyst preparation process, it is also possible to carry out the halogenation treatment prior to reduction in hydrogen. In this case, reduction in hydrogen can take place outside the unit (ex situ), meaning that particular precautions must be taken when transporting the catalyst to that unit, or treatment can take place in the unit (in situ), just before the catalyst is used.

The present invention provides a process for isomerising a feed comprising normal paraffins containing 5 to 8 carbon atoms per molecule as a major portion, characterized in that the sum of the amounts of normal paraffins containing 7 and 8 carbon atoms per molecule contained in the feed is in the range 2% to 90% by weight, preferably in the range 5% to 90% benzene, more preferably in the range 20% to 90% by weight, and highly preferably in the range 40% to 90% by weight with respect to the feed, and in that said feed is treated in at least one reaction zone, preferably containing at least one catalyst in a fixed bed, said catalyst comprising a support, at least one halogen and at least one group VIII metal, the reaction being carried out at a temperature in the range 30° C. to 150° C., preferably 70° C. to 130° C., more preferably in the range 70° C. to 95° C., the feed to be treated preferably containing at least one halogenated compound, more preferably a chlorinated compound, the amount by weight in said feed being in the range 50 to 2000 ppm, usually 50 to 300 ppm, for example perchloroethylene C₂Cl₄.

Two implementations of the invention can be considered, selected depending on the amount of excess hydrogen with respect to the quantity of hydrogen consumed by hydrogenation, naphthene ring opening and paraffin cracking reactions. This can also be expressed as the ratio R of the number of moles of hydrogen over the number of moles of hydrocarbons in the effluent leaving the reactor.

In the first implementation of the invention, a slight excess of hydrogen is used, such that the ratio R of the number of moles of hydrogen over the number of moles of hydrocarbon calculated on the basis of the composition of the effluent leaving the reactor is in the range 0.06 to 0.3, preferably in the range 0.06 to 0.2. In this case it is not necessary to recycle non consumed hydrogen to the reactor inlet. This is thus a “lost hydrogen” operation.

In the second implementation of the process of the invention, a large excess of hydrogen is used. The ratio R of the number of moles of hydrogen over the number of moles of hydrocarbon calculated on the basis of the composition of the effluent leaving the reactor is thus in the range 0.3 to 10, preferably in the range 0.3 to 5, and more preferably in the range 0.5 to 3. In this case the excess hydrogen is recycled to the reactor inlet, for example by means of a gas-liquid separation drum and a recycling compressor. In this implementation of the invention, the range of adjustment for the partial pressure of the hydrogen is wider than in the first implementation.

The preferred ranges given below for the operating conditions are applicable to both implementations of the process of the invention.

The hourly space velocity (HSV), defined as the mass flow rate of feed to be treated per mass of catalyst per hour, is about 0.2 to 10 kg of feed per kg of catalyst per hour (0.2 to 10 h⁻¹), preferably about 0.3 to 5 kg of feed per kg of catalyst per hour (0.3 to 5 h⁻¹), and more preferably about 0.5 to 2 kg of feed per kg of catalyst per hour (0.5 to 2 h⁻¹).

The reactor pressure is about 0.1 to 10 MPa relative, preferably about 0.5 to 8 MPa relative, more preferably between 2 and 5 MPa.

The reactor temperature is in the range 30° C. to 150° C., preferably in the range 70° C. to 130° C., more preferably in the range 70° C. to 95° C.

The use of a catalyst comprising a support, at least one halogen and at least one group VIII metal under the operating conditions described above surprisingly leads to high C5-C8 n-paraffin conversion levels, more particularly n-heptane, while keeping yields of the isomerates high, i.e., the yields of light gasoline essentially constituted by hydrocarbons containing 5 to 8 carbon atoms. The use of this catalyst thus keeps the degree of cracking low.

The process of the invention can be used to treat all types of feeds comprising, as a major portion, normal paraffins containing 5 to 8 carbon atoms, naphthenes, and aromatic compounds (normally in quantities of less than 10% by weight). More particularly, the process of the invention can be used to treat paraffin cuts with a chain containing 5 to 8 carbon atoms, and in which the sum of the amounts of normal paraffins containing 7 and 8 carbon atoms per molecule comprised in the cut is in the range 2% to 90% by weight, preferably in the range 5% to 90% by weight, more preferably in the range 20% to 90% by weight, and highly preferably in the range 40% to 90% by weight.

Preferably, care is taken that feeds for the process of the invention are free of water, oxygen, sulphur and more generally of any known compounds which are known to be poisons or inhibitors for catalysts based on halogenated alumina.

The following examples illustrate the invention without in any way limiting its scope.

### EXAMPLES

The volume of the reactor used was 200 ml, operated in riser mode with the fluids supplied being a mixture constituted by the feed to be treated and hydrogen. The effluent leaving the reactor was cooled then analysed by gas chromatography.

### Example 1 (In Accordance with the Invention)

In this Example, an industrial catalyst based on chlorinate alumina sold by Procatalyse, reference number IS 612A, was used.

The volume of the reactor used was 200 ml, operated in riser mode with the fluids supplied being a mixture constituted by the feed to be treated and hydrogen. The effluent leaving the reactor was cooled then analysed by gas chromatography.

The operating conditions were as follows:

The reactor was fed with a feed comprising hydrocarbons containing 5 to 7 carbon atoms and 800 ppm by weight of perchloroethylene (C₂Cl₄) at a flow rate of 87 g/h, the catalyst mass being 86 g, the HSV 1.01 h⁻¹. The hydrogen flow rate was 4.5×10⁻⁹ l/h. The total pressure was 3 MPa relative. Two isomerisation steps were carried out using the same feed at different temperatures. Isomerisation 1 was carried out at a temperature of 105° C.: the ratio R1 of the number of moles of hydrogen over the number of moles of hydrocarbons, calculated at the reactor outlet, was 0.14; isomerisation 2 was carried out at a temperature of 115° C.: the ratio R2 of the number of moles of hydrogen over the number of moles of hydrocarbons, calculated at the reactor outlet, was 0.11.

The results obtained are shown in Table 1.

TABLE 1

| | | After | After |
|---|---|---|---|
| | Feed | isomerisation 1 | isomerisation 2 |
| Compounds | (wt %) | (wt %) | (wt %) |
| C2-C4 | 0.74 | 5.19 | 7.15 |
| iC5 | 4.19 | 6.72 | 7.62 |
| nC5 | 10.53 | 7.67 | 7.5 |
| Cyclopentane | 0.28 | 0.27 | 0.28 |
| iC6 | 4.01 | 4.32 | 4.73 |
| nC6 | 1.06 | 0.82 | 0.88 |
| Cyclohexane | 1.4 | 3.04 | 2.6 |
| Methylcyclopentane | 1.01 | 1.66 | 1.62 |
| Benzene | 0.01 | 0 | 0 |
| nC7 | 65.7 | 20.00 | 17.35 |
| iC7 | 11.7 | 50.31 | 50.27 |

| | Isomerisation 1 | Isomerisation 2 |
|---|---|---|
| nC5 conversion | 29% | 27% |
| nC6 conversion | 17% | 22.60% |
| nC7 conversion | 73.6% | 69.60% |
| C5 + yield | 93.5% | 95.50% |

The results of Table 1 show that n-heptane conversion levels of the order of 70% were obtained, while producing only 4.45% by weight of light products for isomerisation carried out at 105° C. and 6.41% by weight for isomerisation carried out at 115° C. The term “light products” means a fraction essentially constituted by hydrocarbons containing 2 to 4 carbon atoms.

These results are of great industrial importance as the operating conditions were very mild: temperatures of 105° C. and 115° C.

### Example 2 (In Accordance with the Invention)

This example used the same catalyst and reactor as that of Example 1.

The reactor was supplied with a feed comprising hydrocarbons containing 5 to 7 carbon atoms and 800 ppm of perchloroethylene (C₂Cl₄) at a rate of 84 g/h, the catalyst mass being 84 g, the HSV being 1 h⁻¹. The hydrogen flow rate was 60×10⁻⁹ l/h. The total pressure was 3 MPa relative.

Two isomerisation steps were carried out using the same feed at different temperatures. Isomerisation 3 was carried out at a temperature of 115° C., the ratio R3 of the number of moles of hydrogen over the number of moles of hydrocarbons, calculated at the reactor outlet, was 2.67; isomerisation 4 was carried out at a temperature of 130° C.: the ratio R4 of the number of moles of hydrogen over the number of moles of hydrocarbons, calculated at the reactor outlet, was 2.56.

The principal difference with respect to Example 1 was that Example 2 corresponded to an isomerisation process in which a large excess of hydrogen with respect to the feed to be converted was used.

The composition of the feed and the results obtained are shown in Table 2.

TABLE 2

| | | After | After |
|---|---|---|---|
| | Feed | isomerisation 3 | isomerisation 4 |
| Compounds | (wt %) | (wt%) | (wt%)+TZ 1/32 |
| C2-C4 | 0.87 | 5.99 | 9.51 |
| iCS | 9.95 | 11.73 | 12.5 |
| nC5 | 7.79 | 6.33 | 6.18 |
| Cyclopentane | 0.62 | 0.62 | 0.62 |
| iC6 | 9.50 | 10.40 | 11.01 |
| nC6 | 2.97 | 2.07 | 2.02 |
| Cyclohexane | 5.10 | 3.79 | 3.19 |
| Methylcyclopentane | 2.32 | 2.47 | 2.67 |
| Benzene | 0.17 | 0 | 0 |
| nC7 | 55.41 | 13.63 | 9.15 |
| iC7 | 5.30 | 42.97 | 43.15 |

| | Isomerisation 1 | Isomerisation 2 |
|---|---|---|
| nC5 conversion | 18.8% | 20.7% |
| nC6 conversion | 30.3% | 32% |
| nC7 conversion | 75.4% | 83.5% |
| C5 + yield | 94.8% | 91.3% |

As in Example 1, high degrees of conversion of n-heptanes to iso-heptanes were obtained under operating conditions in which the quantity of light products formed by cracking remained low. The results shown in Table 2 indicate that degrees of n-heptane conversion obtained were of the order of 75-80%, while only 5.1% by weight of light products were produced for isomerisation carried out at 115° C. and 8.7% by weight for isomerisation carried out at 130° C.

Table 2 also shows that 130° C. was a temperature only slightly different from the maximum temperature compatible with the production of high isomerate yields, in particular if it is assumed that the degree of cracking to light products of 10% was the upper acceptable limit. At 130° C., 8.7% of light products were formed by cracking, giving a yield of branched paraffins containing 5 to 7 carbon atoms of 91.3%.

### Example 3 (In Accordance with the Invention)

The catalyst used in Example 3 was produced as follows: gamma alumina was formed by extrusion through a 1.2 mm diameter die. The solid obtained was treated at 500° C. with air containing 3% by weight of steam. 0.2% of platinum was deposited on this alumina by ion exchange with hexachloroplatinic acid in the presence of HCl as a competing agent. The solid obtained was reduced in hydrogen at 400° C.

The solid obtained was then chlorinated at a temperature of 280° C. by injecting carbon tetrachloride in a stream of nitrogen.

The feed to be treated was constituted by about 10% by weight of normal paraffins containing 5 carbon atoms, 10% by weight of normal paraffins containing 6 carbon atoms, 65% by weight of normal paraffins containing 7 carbon atoms and 8% by weight of naphthenes containing 6 carbon atoms. The feed containing 100 ppm of carbon tetrachloride (CCl₄), expressed as the weight of chlorine, to maintain the amount of chlorine in the catalyst used.

The isomerisation operating conditions were as follows: the reactor temperature was 110° C., the total pressure was 3 MPa relative, the HSV was 1 h⁻¹ and the ratio R5 of the number of moles of hydrogen over the number of moles of hydrocarbons calculated at the reactor outlet was 0.47.

The performances obtained after 24 hours of operation were as follows: a degree of n-heptane conversion of 73.5, and only 4.6% by weight of light products were produced.

1. A process for isomerising a feed comprising normal paraffins containing 5 to 8 carbon atoms per molecule as a major portion in the presence of hydrogen, characterized in that the sum of the amounts of normal paraffins containing 7 and 8 carbon atoms per molecule contained in the feed is in the range 2% to 90% by weight with respect to the feed, and in that said feed is treated in at least one reaction zone, containing at least one catalyst in a fixed bed, said catalyst comprising a support, at least one halogen and at least one group VIII metal, the reaction being carried out at a temperature in the range 30 to 150° C.

2. A process according to claim 1, in which the support is alumina based.

3. A process according to claim 1 or claim 2, in which the feed to be treated contains at least one halogenated compound in an amount in said feed in the range 50 to 2000 ppm by weight.

4. An isomerisation process according to any one of claims 1 to 3, characterized in that the halogen contained in the support is chlorine.

5. An isomerisation process according to any one of claims 1 to 4, characterized in that a treatment of the support at high temperature in steam is carried out before or after depositing at least one metal.

6. An isomerisation process according to claim 5, in which the support is treated for 0.5 to 6 hours at a temperature of about 200° C. to 700° C., in a stream of a gas containing water in amounts of about 0.2% to 100% by volume.

7. An isomerisation process according to any one of claims 1 to 6, in which the sum of the amounts of normal paraffins containing 7 and 8 carbon atoms per molecule contained in the feed is in the range 5% to 90% by weight.

8. An isomerisation process according to any one of claims 1 to 6, in which the sum of the amounts of normal paraffins containing 7 and 8 carbon atoms per molecule contained in the feed is in the range 20% to 90% by weight.

9. An isomerisation process according to any one of claims 1 to 8, characterized in that the support contains a halogen in amounts in the range 0.1% to 15% by weight.

10. An isomerisation process according to any one of claims 1 to 9, characterized in that the total reaction pressure is about 0.1 to 10 MPa relative, the hourly space velocity being about 0.2 to 10 h⁻¹.

11. An isomerisation process according to any one of claims 1 to 10, characterized in that the reaction is carried out in the presence of an excess of hydrogen such that the ratio R of the number of moles of hydrogen over the number of moles of hydrocarbons calculated on the basis of the composition of the effluent leaving the reactor is in the range 0.06 to 0.3.

12. An isomerisation process according to any one of claims 1 to 10, characterized in that the reaction is carried out in the presence of an excess of hydrogen such that the ratio of the number of moles of hydrogen over the number of moles of hydrocarbons calculated on the basis of the composition of the effluent leaving the reactor is in the range 0.3 to 10.

13. An isomerisation process according to any one of claims 1 to 12, characterized in that the catalyst undergoes treatment in hydrogen before depositing at least one halogen.

14. An isomerisation process according to claim 13, characterized in that the treatment in hydrogen comprises a slow rise in temperature in a stream of hydrogen up to the maximum reduction temperature which is about 300° C. to 700° C., followed by maintaining that temperature, generally for 1 to 6 hours.
